(19) United States
(12) Patent Application Publication
(10) Pub. No.: US 2025/0221644 A1
(43) Pub. Date: Jul. 10, 2025

(54) CALIBRATION FREE ANALYTE DETECTION DEVICE

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/695,366

(22) PCT Filed: Jan. 30, 2022
(86) PCT No.: PCT/CN2022/075204

§ 371 Date: Mar. 26, 2024

(30) Foreign Application Priority Data
Sep. 27, 2021 (WO) PCT/CN2021/120856

(51) Int. Cl. A61B 5/1495; A61B 5/00; A61B 5/145

(52) U.S. Cl. CPC A61B 5/1495; A61B 5/14503; A61B 5/14532; A61B 5/6847; A61B 5/7264; A61B 5/746; A61B 2560/0214; A61B 2560/0228; A61B 2560/0443; A61B 2560/0462

(57) ABSTRACT

A calibration free analyte detection device (102, 202, 302) is disclosed. At least one predetermined pair-data set composed of the first parameter value and the second parameter value and the predetermined calibration function based on the time parameter are pre stored in the memory. After a sensor (1022, 2022, 3022) penetrates into the user's skin to obtain the first parameter value, the processor calls the predetermined pair-data set and the predetermined calibration function from the memory and obtains the second parameter value based on the first parameter value by index, the predetermined pair-data set is adjusted through the predetermined calibration function for the next detection and retrieval. The predetermined pair-data set is adjusted with the use time, which reduces the error of the second parameter value and improves the reliability of the analyte detection device (102, 202, 302).

### TECHNICAL FIELD

The invention mainly relates to the field of medical device, in particular to a calibration free analyte detection device.

### BACKGROUND

The pancreas in a normal human body can automatically monitor the blood glucose level and automatically secrete required amount of insulin/glucagon. In the body of a type 1 diabetes patient, the pancreas does not function properly and cannot produce enough insulin for the body. Therefore, type 1 diabetes is a metabolic disease caused by abnormal pancreatic function, and diabetes is a lifelong disease. At present, there is no cure for diabetes with medical technology. The onset and development of diabetes and its complications can only be controlled by stabilizing blood glucose.

Diabetics need to have their blood glucose measured before they inject insulin into the body. At present, most of the testing methods can continuously measure blood glucose level and transmit the data to a remote equipment in real time for the user to view. This method is called Continuous Glucose Monitoring (CGM).

The prior art analyte detection device is calibrated before or during use. The method adopted is to linearly adjust the sensitivity of the sensor based on the predetermined calibration function. However, in the actual use process, the sensitivity of the sensor does not change linearly, resulting in the low reliability of the analyte parameter information detected by the sensor. Therefore, the prior art urgently needs a more reliable analyte detection device.

### BRIEF SUMMARY OF THE INVENTION

The embodiment of the invention discloses a calibration free analyte detection device. At least one predetermined pair-data set composed of the first parameter value and the second parameter value and the predetermined calibration function based on the time parameter are pre stored in the memory. After the sensor penetrates into the user's skin to obtain the first parameter value, the processor calls the predetermined pair-data set and the predetermined calibration function from the memory and obtains the second parameter value based on the first parameter value by index, the predetermined pair-data set is adjusted through the predetermined calibration function for the next detection and retrieval. The predetermined pair-data set is adjusted with the use time, which reduces the error of the second parameter value and improves the reliability of the analyte detection device.

The invention discloses an analyte detection device, which comprises: the shell; The sensor comprises an internal part and an external part, and the internal part is used to penetrate into the user's subcutaneous skin to obtain the first parameter value; The memory in which at least a predetermined pair-data set composed of the first parameter value and the second parameter value and the predetermined calibration function based on time parameters are pre stored, and the second parameter value is associated with the in vivo analyte parameter information; The processor is programmed to call the predetermined pair-data set and the predetermined calibration function from the memory, and then obtain the second parameter value based on the first parameter value by index, and the predetermined pair-data set is adjusted by predetermined calibration function, which can be called by the processor in the next detection; The transmitter is used to transmit the first parameter value and/or the second parameter value to the remote equipment; And a battery, which is used to provide electric energy.

According to one aspect of the invention, the first parameter value is current value or voltage value.

According to one aspect of the invention, the second parameter value comprises at least the blood glucose concentration value.

According to one aspect of the invention, the predetermined pair-data set is at least partially derived from in vivo tests.

According to one aspect of the invention, the predetermined pair-data set is at least partially derived from in vitro tests.

According to one aspect of the invention, at least part of the pair-data in the predetermined pair-data set can also be adjusted based on the physical characteristics of the sensor.

According to one aspect of the invention, the physical characteristics of the sensor comprise at least one of the membrane thickness, active enzyme area, active enzyme volume or resistance of the electrode.

According to one aspect of the invention, the adjustment of pair-data is fixed.

According to one aspect of the invention, the adjustment of pair-data is linear.

According to one aspect of the invention, the first parameter value or the second parameter value is set with at least one threshold, when the first parameter value or the second parameter value exceeds the threshold, the remote equipment sends an alert indication.

According to one aspect of the invention, the threshold is set by the user or non-user.

According to one aspect of the invention, the second parameter value is obtained by interpolating the first parameter value.

According to one aspect of the invention, the first parameter value in the predetermined pair-data set is a continuous range interval, and the second parameter value is obtained by judging the range interval in which the first parameter value falls with indexing.

According to one aspect of the invention, the adjustment of the predetermined pair-data set includes the adjustment of the first parameter value and/or the second parameter value.

According to one aspect of the invention, the transmitter, memory, sensor, processor and battery are located in the shell.

According to one aspect of the invention, the transmitter, sensor and battery are located in the shell, and the memory and/or processor are located in the remote equipment.

According to one aspect of the invention, at least two of the transmitter, processor or memory are integrated into one device.

The invention also discloses a calibration method for analyte detection, which comprises: provide the memory, at least a predetermined pair-data set composed of the first parameter value and the second parameter value and the predetermined calibration function based on time parameters are pre stored in the memory, and the second parameter value is associated with the in vivo analyte parameter information; The sensor comprises an internal part and an external part, and the internal part is penetrated into the user's subcutaneous to obtain the first parameter value; The processor is programmed to obtain the first parameter value from the sensor, call the predetermined pair-data set and the predetermined calibration function from the memory, and then obtain the second parameter value based on the first parameter value by index, and the predetermined pair-data set is adjusted by predetermined calibration function, which can be called by the processor in the next detection; The transmitter sends the first parameter value and/or the second parameter value to the remote equipment.

Compared with the prior art, the technical scheme of the invention has the following advantages:

In the analyte detection device disclosed by the invention, at least one predetermined pair-data set composed of the first parameter value and the second parameter value and the predetermined calibration function based on the time parameter are pre stored in the memory. After the sensor penetrates into the user's skin to obtain the first parameter value, the processor calls the predetermined pair-data set and the predetermined calibration function from the memory and obtains the second parameter value based on the first parameter value by index, the predetermined pair-data set is adjusted through the predetermined calibration function for the next detection and retrieval. The predetermined pair-data set is adjusted with the use time, which reduces the error of the second parameter value and improves the reliability of the analyte detection device.

Further, at least part of the pair-data of the predetermined pair-data set can be adjusted according to the physical characteristics of the sensor, so as to improve the accuracy of obtaining the second parameter value and improve the reliability of the analyte detection device.

Further, the second parameter value is obtained from the first parameter value through interpolation operation, which avoids the failure of the index of the first parameter value and improves the reliability of the predetermined pair-data set.

Further, the first parameter value in the predetermined pair-data set is a continuous range interval, and the second parameter value is obtained by judging the range interval in which the first parameter value falls with indexing, so as to avoid the failure of indexing and improve the reliability of the analyte detection device.

Further, the adjustment of the pair-data is fixed or linear. The pair-data can be flexibly adjusted according to the actual needs to improve the accuracy of obtaining the second parameter value and improve the reliability of the analyte detection device.

Further, the first parameter value or the second parameter value is set with at least one threshold, such as hyperglycemia and/or hypoglycemia threshold. When any parameter value exceeds the threshold, a high/low blood glucose alert will be triggered to prompt the user of blood glucose risk, so as to improve the user experience.

Further, the memory and/or processor are located in remote equipment, which can improve the storage capacity of the memory and the calculation capacity of the processor.

Further, at least two of the memory, the processor or the transmitter are integrated into one device, which is conducive to reducing the occupied volume of the device and facilitating the miniaturization design of the analyte detection device.

### BRIEF DESCRIPTION OF THE DRAWINGS

FIG. 1 is a schematic diagram of the structure of the analyte detection system according to an embodiment of the invention;

FIG. 2 is a schematic diagram of the analyte detection device according to an embodiment of the invention;

FIG. 3a is a structural schematic diagram of the wake-up module of the analyte detection device comprising a sensor according to an embodiment of the invention;

FIG. 3b is a schematic diagram of the wake-up module of the analyte detection device comprising a light sensing element according to an embodiment of the invention;

FIG. 4a is a structural schematic diagram of the analyte detection system comprising magnetic component and magnetic induction element according to an embodiment of the invention;

FIG. 4b is a structural schematic diagram of the wake-up module of an analyte detection device comprising a magnetic induction element according to an embodiment of the invention;

FIG. 4c is a schematic diagram of the wake-up module of the analyte detection device comprising a magnetic induction element according to an embodiment of the invention;

FIG. 5a is a structural schematic diagram of the analyte detection system comprising an acceleration sensor according to an embodiment of the invention;

FIG. 5b is a structural schematic diagram of the wake-up module of the analyte detection device comprising an acceleration sensor according to an embodiment of the invention;

FIG. 5c is a functional schematic diagram of the wake-up module of the analyte detection device comprising the acceleration sensor to an embodiment of the invention.

FIG. 6a-6b are two kinds of structural diagrams of the sensor according to an embodiment of the invention;

FIG. 7 is the first pair-data set according to an embodiment of the invention;

FIG. 8 is the schematic diagram of communication between an analyte detection device and the remote equipment according to an embodiment of the invention;

FIG. 9 is the flow chart of using the first pair-data set according to an embodiment of the invention;

FIG. 10 is the second pair-data set according to an embodiment of the invention;

FIG. 11 is the flow chart of using the second pair-data set according to an embodiment of the invention;

FIG. 12 is the flow chart of the first calibration scheme according to an embodiment of the invention;

FIG. 13a is the average pair-data set according to an embodiment of the invention;

FIG. 13b is the average range value pair-data set according to an embodiment of the invention;

FIG. 14 is the flow chart of the second calibration scheme according to an embodiment of the invention;

FIG. 15a is the flow chart of calibration based on differences in physical characteristics of the sensor according to an embodiment of the invention;

FIG. 15b is the schematic diagram of the visualization curve of the pair-data set in a coordinate system according to an embodiment of the invention;

FIG. 16 is the schematic diagram of the pair-data set in a second calibration scheme according to an embodiment of the invention;

FIG. 17 is the first flow chart of calibrating based on time parameter difference according to an embodiment of the invention;

FIG. 18 is the second flow chart of calibrating based on time parameter difference according to an embodiment of the invention.

### DETAILED DESCRIPTION

As mentioned above, the analyte detection device in prior art is calibrated before or during use. The method adopted is to linearly adjust the sensitivity of the sensor based on the predetermined calibration function, but in the actual use process, the sensitivity of the sensor does not change linearly. At the same time, with the increase of the use time of the sensor, the sensitivity of the sensor is gradually changing due to the change of enzyme layer activity, electrode oxidation and other factors, as a result, the reliability of analyte parameter information detected by the sensor is not high.

In order to solve this problem, the invention provides a calibration free analyte detection device. At least one predetermined pair-data set composed of the first parameter value and the second parameter value and the predetermined calibration function based on the time parameter are pre stored in the memory. After the sensor penetrates into the user's skin to obtain the first parameter value, the processor calls the predetermined pair-data set and the predetermined calibration function from the memory and obtains the second parameter value based on the first parameter value by index, the predetermined pair-data set is adjusted through the predetermined calibration function for the next detection and retrieval. The predetermined pair-data set is adjusted with the use time, which reduces the error of the second parameter value and improves the reliability of the analyte detection device.

Various exemplary embodiments of the invention will now be described in detail with reference to the attached drawings. It is understood that, unless otherwise specified, the relative arrangement of parts and steps, numerical expressions and values described in these embodiments shall not be construed as limitations on the scope of the invention.

In addition, it should be understood that the dimensions of the various components shown in the attached drawings are not necessarily drawn to actual proportions for ease of description, e. g. the thickness, width, length or distance of some elements may be enlarged relative to other structures.

The following descriptions of exemplary embodiments are illustrative only and do not in any sense limit the invention, its application or use. Techniques, methods and devices known to ordinary technicians in the relevant field may not be discussed in detail here, but to the extent applicable, they shall be considered as part of this manual.

It should be noted that similar labels and letters indicate similar items in the appending drawings below, so that once an item is defined or described in one of the appending drawings, there is no need to discuss it further in the subsequent appending drawings.

FIG. 1 is the structural diagram of the analyte detection system in an embodiment of the invention. The analyte detection system 10 comprises an auxiliary installer 101 and an analyte detection device 102. The auxiliary installer 101 comprises a housing 1011 and an auxiliary mounting module 1012, which is located inside the housing 1011. Analyte detection device 102 is located at the ejector end of auxiliary mounting module 1012, which enables rapid installation of analyte detection device 102 to the host skin surface when in use.

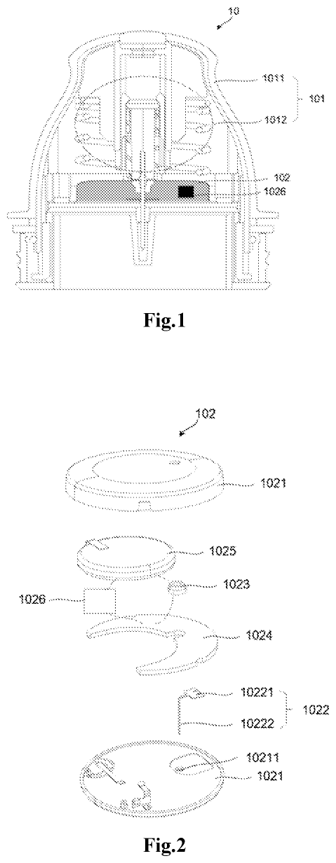

FIG. 2 is the schematic diagram of the analyte detection device to an embodiment of the invention. The analyte detection device 102 comprises the shell 1021, the sensor 1022, the transmitter 1023, the internal circuit 1024, the battery 1025, the wake-up module 1026, the memory 1027 and the processor 1028. Sensor 1022 comprises an external part 10221 and an internal part 10222. The external part 10221, transmitter 1023, internal circuit 1024, battery 1025 and wake-up module 1026 are located inside the shell 1021. The internal part 10222 passes through the through hole 10211 on the shell 1021 to the outside to puncture the host subcutaneous and detect the parameter information of analyte. What technicians in this field can know is that in order to pierce the internal part 10222 subcutaneous to the host, the through hole 10211 is located on the side of shell 1021 which is away from housing 1011, and at the same time, a tape (not shown in the figure) is arranged on the surface, which is used to attach the analyte detection device 102 to the skin surface of the host. The external part 10221 is electrically connected with the transmitter 1023 through the internal circuit 1024, which can transmit analyte parameter information to the remote equipment.

Before use, the shell 1021 of the analyte detection device 102 is releasable connected with the housing 1011 of auxiliary mounting device 101. Here, “releasable connection” means that shell 1021 is connected with housing 1011 by means of buckle, clamp, etc. Under the action of ejector mechanism of auxiliary mounting module 1012, the shell 1021 can be separated from housing 1011.

After the life of the sensor 1022 has expired, or the battery 1025 has run out of power, or other factors have caused the analyte detection device to fail, the user removes the entire analyte detection device from the skin surface of the host, discards it and replaces it with a new analyte detection device, is beneficial to maintain the best use of the parts of the device.

When analyte detection device 102 is installed on the skin surface of the host and starts to use, communication needs to be established with remote equipment such as PDM (Personal Diabetes Manager), mobile phone, etc., for data interaction, so as to transmit the detected analyte information data in the host to remote equipment.

As mentioned above, the analyte detection device 102 is in dormant state and transmits signal to the remote equipment at the first frequency until communication is formally established with the remote equipment. In the embodiment of the invention, the analyte detection device 102 transmits signal at a lower first frequency to the remote equipment in dormant state to reduce battery energy consumption. In the more preferred embodiment of the invention, the first frequency is 0˜12 times/hour. In the more preferred embodiment of the invention, the first frequency is 0 times/hour, that is, the analyte detection device 102 does not transmit signal to the remote equipment in dormant state.

In order to establish communication between the analyte detection device 102 which is in dormant state and remote equipment, wake-up module 1026 wakes up analyte detection device 102 according to triggering conditions, so that it enters the working state and transmits signal to the remote equipment with the second frequency, and then communication is established after the remote equipment responds. The second frequency is higher than the first frequency in order to obtain analyte parameter information conveniently and in real time. In the preferred embodiment of the invention, the second frequency is 12˜3600 times/hour. In a more preferred embodiment of the invention, the second frequency is 30 times/hour.

### First Embodiment

### Light Sensing Element

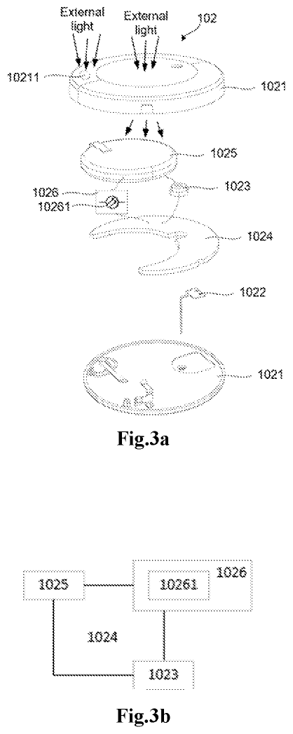

FIG. 3a is a schematic diagram of the structure of the wake-up module of the analyte detection device comprising a light sensing element in an embodiment of the invention. FIG. 3b is a functional schematic diagram of the wake-up module of the analyte detection device comprising the light sensing element in an embodiment of the invention.

In the embodiment of the invention, the wake-up module 1026 comprises a light sensing element 10261, such as photoelectric switch, which is in open state when there is no light beam or weak light beam irradiation and in a closed state when there is light beam irradiation.

In combination with FIG. 1 and FIG. 3b, transmitter 1023 is connected with battery 1025 through internal circuit 1024, forming a closed loop. The circuit is connected with a wake-up module 1026, which is connected with a light sensing element 10261 inside. The triggering condition of the wake-up module 1026 is the light intensity change received by the light sensing element 10261. In the preferred embodiment of the invention, the triggering condition of the wake-up module 1026 is that the light intensity received by the light sensing element 10261 changes from weak to strong.

In the embodiment of the invention, the analyte detection device 102 is not separated from the auxiliary mounting device 101 before it is installed on the skin surface of the host, and the shell 1021 and housing 1011 form a closed and opaque space. Since the light-transmitting area 10211 is located near the end of the housing 1011, there is no external light irradiates on light sensing element 10261, battery 1025 supplies power to transmitter 1023 through wake-up module 1026 (comprising light sensing element 10261), light sensing element 10261 is in open state, and thus the transmitter 1023 is in dormant state, and analyte detection device 102 transmits signal to remote equipment at the first frequency. After the analyte detection device 102 is installed on the skin surface of the host through the auxiliary mounting module 1012, the shell 1021 is separated from the housing 1011, and the external light can be irradiated to the light sensing element 10261 through the shell 1021. The light sensing element 10261 is in closed state. The transmitter 1023 enters the working state, and the analyte detection device 102 transmits signal to the remote equipment at the second frequency. After the response of the remote equipment, the communication is established and the analyte detection data is transmitted to the remote equipment.

In the embodiment of the invention, the shell 1021 is made of light transmittance material, such as one of polymethyl methacrylate (PMMA), polystyrene (PS), polycarbonate (PC) or poly 4-methyl-1-pentene (TPX), and the light transmittance of these material is 40%˜95%. After the separation of shell 1021 and housing 1011, the external light can be irradiated on the light sensing element 10261 through the shell 1021.

In other embodiment of the invention, the shell 1021 comprises light-transmitting area 10211, the light transmittance of the light-transmitting area 10211 is higher than that of the shell 1021, so that more external light is irradiated on the light sensing element 10261, the light intensity variation of the light sensing element 10261 is increased, and the reliability of the light sensing element 10261 is improved.

In another embodiment of the invention, the light-transmitting area 10211 comprises at least one light-transmitting hole, or an array combination of several light-transmitting holes. The light-transmitting hole can make more external light illuminate on the light sensing element 10261, further increase the light intensity variation of the light sensing element 10261, and improve the reliability of the light sensing element 10261. A light-transmittance film is arranged in the light-transmitting hole (not shown in the figure out), which can prevent external water droplets, dust and other dirt from entering the analyte detection device through the light-transmitting hole and improve the reliability of the device.

In the embodiment of the invention, the light sensing element 10261 can sense visible light or invisible light, such as infrared or ultraviolet light. In the preferred embodiment of the invention, the light sensing element 10261 senses visible light so that the user can wake up the analyte detection device indoors or outdoors.

In other embodiment of the invention, the switch condition of open circuit and closed circuit of the light sensing element is low light irradiation to strong light irradiation, that is, before the separation of shell 1021 and housing 1011, weak external light is allowed to illuminate the interior of housing 1011, and the light sensing element 10261 receives weak light, but it is still in open circuit and the transmitter 1023 is in dormant state, which takes into account that the actual connection between shell 1021 and housing 1011 is not completely sealed. When the shell 1021 is separated from the housing 1011, the external light completely irradiates on the light sensing element 10261 through the shell 1021, and the light intensity received by the light sensing element 10261 becomes stronger. After reaching the set light intensity threshold, the light sensing element 10261 switches to the closed state, and the transmitter 1023 enters the working state to transmit signal to the remote equipment at the second frequency. After the response from the remote equipment, the communication is established and the analyte detection data is transmitted to the remote equipment.

### Second Embodiment

### Magnetic Component and Magnetic Induction Element

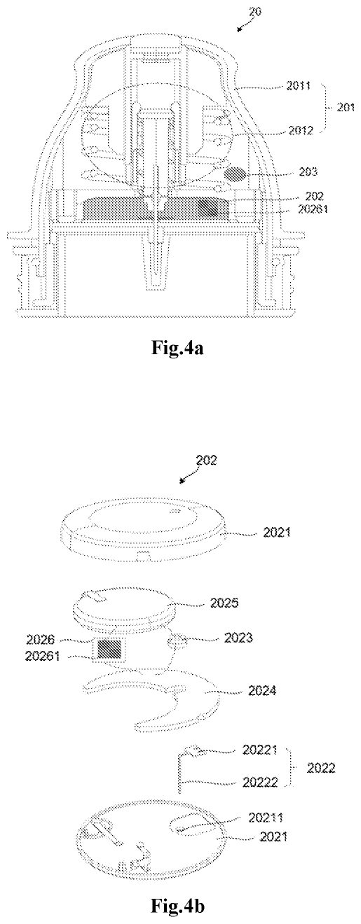

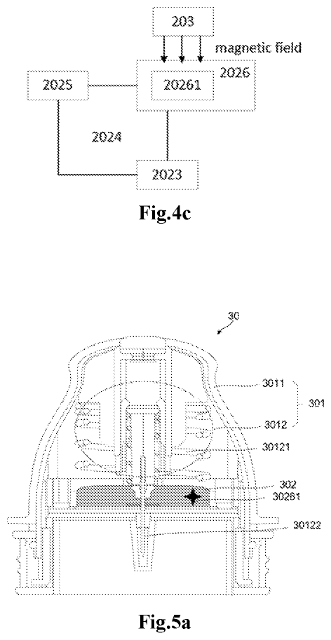

FIG. 4a is a schematic diagram of the structure of the analyte detection system comprising magnetic component and magnetic induction element in an embodiment of the invention. FIG. 4b is a schematic diagram of the structure of the wake-up module of the analyte detection device comprising the magnetic induction element in an embodiment of the invention. FIG. 4c is a schematic diagram of the function of the wake-up module of the analyte detection device comprising the magnetic induction element in an embodiment of the invention.

In the embodiment of the invention, a magnetic component 203 is arranged on the housing 2011, and a magnetic induction element 20261 is arranged in the wake-up module 2026, the battery 2025 supplies power to transmitter 2023 through the wake-up module 2026 (comprising the magnetic induction element 20261). Magnetic component 203 provides a stable magnetic field, and magnetic induction element 20261 is located in the magnetic field of magnetic component 203 and induces the magnetic field of magnetic component 203 to generate a signal. The triggering condition of the wake-up module 2026 is the magnetic field change induced by the magnetic induction element 20261.

The transmitter 2023 is connected with the battery 2025 through the internal circuit 2024, forming a closed loop, and the circuit is connected with the wake-up module 2026. Before the analyte detection device 202 is installed on the skin surface of the host, the analyte detection device 202 is not separated from the auxiliary mounting device 201, and the relative position is fixed. The magnetic field induced by the magnetic induction element 20261 to the magnetic component 203 is stable. Under the stable magnetic field, the magnetic induction element 20261 is in the open state, the transmitter 2023 is in dormant state, and analyte detection device 202 transmits signal to remote equipment at the first frequency. After the analyte detection device 202 is installed on the skin surface of the host through the auxiliary mounting module 2012, the shell 2021 is separated from the housing 2011, and the distance between the magnetic induction element 20261 and the magnetic component 203 changes, so the induced magnetic field also changes, and the magnetic induction element 20261 switches to the closed state, and transmitter 2023 enters the working state. Analyte detection device 202 transmits signal to the remote equipment at the second frequency, and then establishes communication with remote equipment after the response of the remote equipment, and transmits analyte detection data to the remote equipment.

In the embodiment of the invention, the magnetic induction element 20261 senses the magnetic field strength or magnetic field direction of the magnetic component 203. Preferably, the induction element 20261 comprises a hall element (not shown in the figure out) that sensitively sensitizes the magnetic field strength of the magnetic component 203.

In the embodiment of the invention, the magnetic component 203 may be an individual part independent of the housing 2011, or a part of the housing 2011 which is embedded in the housing 2011.

In other embodiments of the invention, the housing 2011 is embedded or enclosed with a magnetic field shielding device (not shown in the figure out), such as a Faraday cage. Technicians in this field can know that the magnetic shielding device is located outside the magnetic component 203 to reduce the impact of external magnetic field on the magnetic induction element 20261.

### Third Embodiment

### Acceleration Sensor

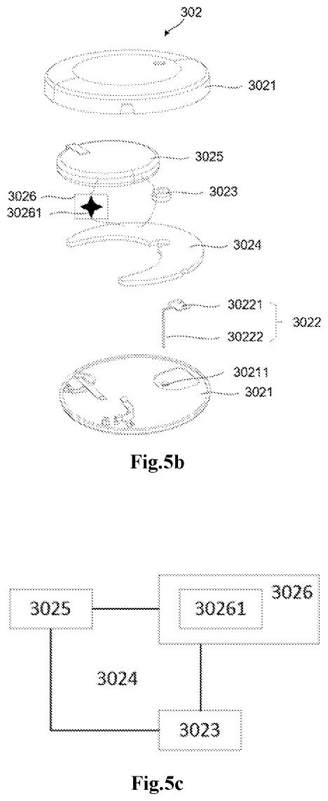

FIG. 5a is a schematic diagram of the structure of the wake-up module of the analyte detection system comprising the acceleration sensor in an embodiment of the invention. FIG. 5b is a schematic diagram of the structure of the wake-up module of the analyte detection device comprising the acceleration sensor in an embodiment of the invention. FIG. 5c is a schematic diagram of the function of the wake-up module of the analyte detection device comprising the acceleration sensor in an embodiment of the invention.

In the embodiment of the invention, the wake-up module 3026 comprises an acceleration sensor 30261, which can sensitively sense the values of motion parameters such as acceleration and adjust the circuit state of the wake-up module 3026 accordingly. The triggering condition of wake-up module 3026 is the motion parameter change of acceleration sensor 30261.

Transmitter 3023 is connected with battery 3025 through internal circuit 3024 to form a closed loop, and the circuit is connected with the wake-up module 3026, the battery 3025 supplies power to transmitter 3023 through wake-up module 3026 (comprising acceleration sensor 30261). Before the analyte detection device 302 is installed on the skin surface of the host, the analyte detection device 302 and the auxiliary mounting device 301 are relatively fixed. In order to pierce the internal part of the sensor 30222 of the analyte detection device into the skin of the host and reduce the pain sensation during the stabbing, the auxiliary mounting module 3012 adopts ejector mechanism 30121. Such as spring and other elastic parts, through the auxiliary needle 30122 can quickly pierce the body part 30222 into the host subcutaneous. When the ejector mechanism 30121 is in use, it produces a large instantaneous forward acceleration a1, and when it is installed on the skin surface of the host, it is obstructed by the skin to produce a reverse acceleration a2. After the acceleration sensor 30261 senses the above two accelerations, it can be determined that the analyte detection device 302 is installed on the skin surface of the host.

In the embodiment of the invention, before the analyte detection device 302 is installed on the skin surface of the host, the wake-up module 3026 is in an open state, and the transmitter 3023 is in a dormant state and transmits signal to the remote equipment at the first frequency. Acceleration sensor 30261 determines that the analyte detection device 302 is installed on the skin surface of the host, and the wake-up module 3026 switches to the closed state, and transmitter 3023 enters the working state and transmits signal to the remote equipment at the second frequency. After the response of the remote equipment, the communication is established and the analyte detection data is transmitted to the remote equipment.

Continue to refer to FIG. 2. In the embodiment of the invention, the sensor 1022 comprises an external part 10221 and an internal part 10222. The external part 10221 is tiled on the inner side of the shell 1021, which can reduce the height of the sensor and reduce the thickness of the analyte detection system. The internal part 10222 is bent relative to the external part 10221 and passes through the through hole 10211 on the shell 1021 to the outside.

In the preferred embodiment of the invention, the internal part 10222 is bent 90° relative to the external part 10221.

In the embodiment of the invention, the internal part 10222 is penetrated into the user's skin to obtain the first parameter value, and the external part 10221 is electrically connected with the internal circuit 1024.

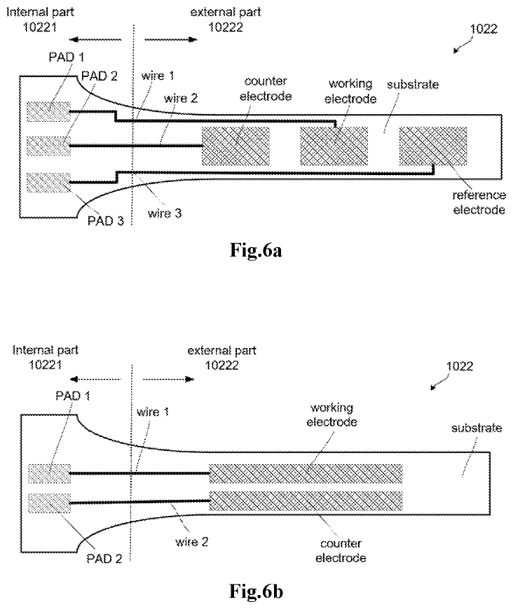

FIG. 6a and FIG. 6b are two kinds of structural diagrams of the sensor according to the embodiment of the invention.

Referring to FIG. 6a, in the embodiment of the invention, the internal part 10222 comprises two electrodes, namely working electrode and counter electrode, which are electrically connected with PAD 1 and PAD 2 through wire 1 and wire 2 respectively. PAD 1 and PAD 2 are arranged on the external part 10221 and electrically connected with the internal circuit 1024. Electrodes, wires and PADs are fixed on the insulating substrate.

Referring to FIG. 6b, in the embodiment of the invention, the internal part 10222 comprises three electrodes, namely working electrode, counter electrode and reference electrode, which are electrically connected with PAD 1, PAD 2 and PAD 3 through wire 1, wire 2 and wire 3 respectively. PAD 1, PAD 2 and PAD 3 are arranged on the external part 10221 and electrically connected with the internal circuit 1024. Electrodes, wires and PADs are fixed on the insulating substrate.

In the embodiment of the invention, an active enzyme layer capable of reacting with an analyte in vivo is also arranged on the electrode. For example, when the analyte in vivo is glucose, the active enzyme layer is glucose active enzyme. When the glucose active enzyme is in contact with glucose in vivo, different numbers of electrons will be generated according to the different glucose concentration ƒ(xₙ) (second parameter value), so as to form different current value or voltage value xₙ (first parameter value) on the electrode. That is, the predetermined pair-data of the first parameter value and the second parameter value can be obtained, and when using the sensor 1022, the second parameter value can be obtained according to the first parameter value.

In the preferred embodiment of the invention, the first parameter value is the current value of the sensor 1022.

In other embodiments of the invention, the in vivo analyte can also be adrenaline, thyroid hormone, hemoglobin or other in vivo substances, which are not limited here.

In other embodiments of the invention, the second parameter value can also be other parameters of the analyte in vivo, such as the type of analyte, etc.

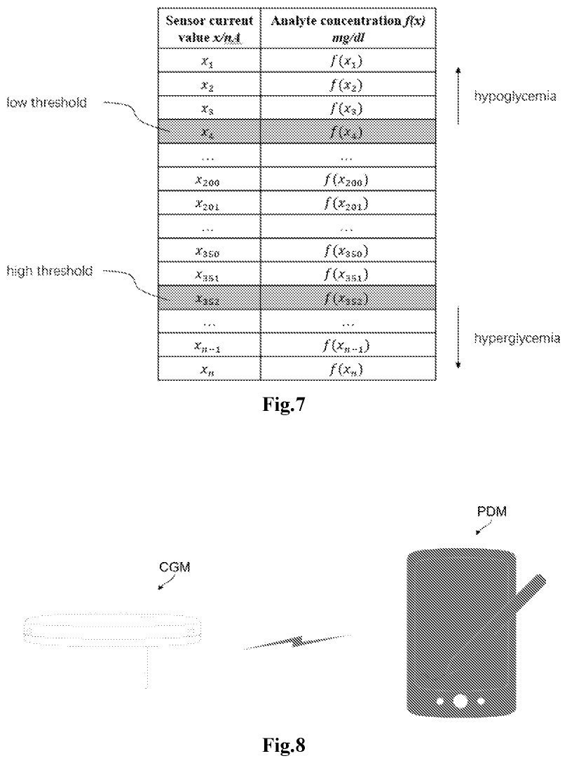

In some embodiments of the invention, the predetermined pair-data of the first parameter value and the second parameter value can be obtained in vitro. For example, the sensor 1022 is placed in an analyte solution with different concentrations ƒ(xₙ) (second parameter value), the standard operating voltage is provided, and the feedback current value xₙ (first parameter value) of the sensor 1022 is measured to obtain the first pair-data set as shown in FIG. 7.

In other embodiments of the invention, the predetermined pair-data of the first parameter value and the second parameter value can be obtained in vivo. For example, the sensor 1022 is penetrated into the user's body, the standard working voltage is provided, the analyte concentration ƒ(xₙ) (second parameter value) in the body is obtained by using finger blood, etc., and the feedback current value xₙ (first parameter value) of the sensor 1022 is measured to obtain the pair-data set. When the predetermined pair-data is obtained from in vivo test, the interference of other analytes and environmental factors on the first parameter value can be eliminated, and the predetermined pair-data obtained from the test owns higher accuracy.

In other embodiments of the invention, the predetermined pair-data set of the first parameter value and the second parameter value can be obtained by testing in vivo and in vitro at same time.

In the preferred embodiment of the invention, the predetermined pair-data of the first parameter value and the second parameter value are obtained in vitro. During in vitro test acquisition, the accuracy and range of the second parameter value can be manually controlled, so as to obtain the pair-data set with higher accuracy and wider range.

In some embodiments of the invention, during the test, the range of analyte concentration ƒ(xₙ) is preset to be 30 mg/dL˜150 mg/dL, and the accuracy is 0.1 mg/dL, for example, when ƒ(x₁)=30 mg/dL, ƒ(x₂)=30.1 mg/dL . . . and the current values x₁, x₂ . . . of the sensor 1022 at the corresponding concentration are obtained respectively, so as to obtain 1201 pair-data sets. In other embodiments of the invention, during the test, the range of analyte concentration ƒ(xₙ) is preset to be 10 mg/dL˜200 mg/dL, and the accuracy is 1 mg/dL, for example, when ƒ(xₙ)=10 mg/dL, ƒ(x₂)=11 mg/dL . . . and the current values x₁, x₂ . . . of sensor 1022 at the corresponding concentration are obtained respectively, so as to obtain the 191 pair-data sets. Considering the storage space of the memory in the analyte detection system and the actual use requirements, different test ranges and test accuracy can be set.

In the embodiment of the invention, after the pair-data set of the first parameter value and the second parameter value is tested, the pair-data set is input into the memory of the analyte detection system for calling and indexing when in use.

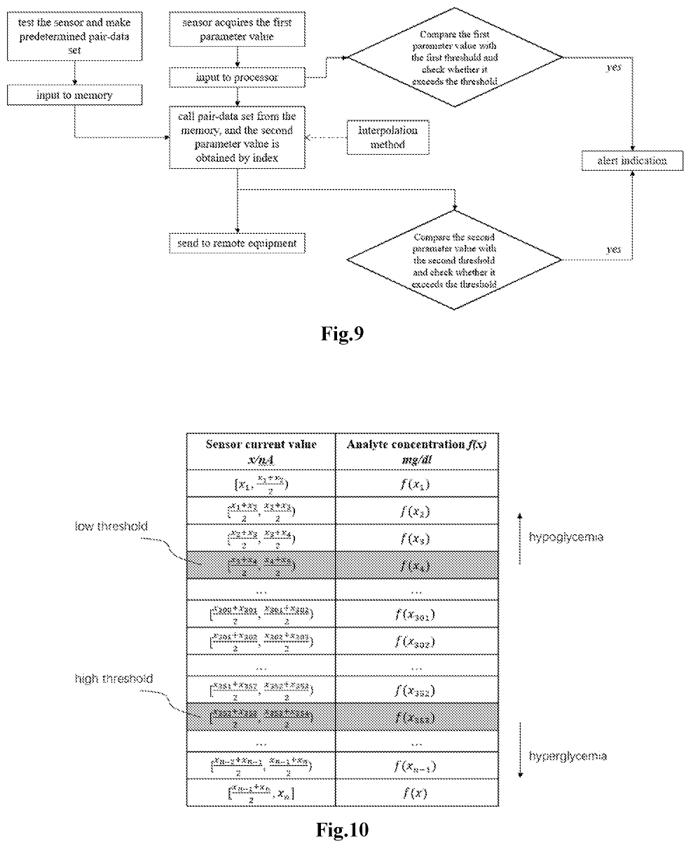

FIG. 8 is the schematic diagram of analyte detection system. FIG. 9 is the flow chart of the use of the first pair-data set of the analyte detection system.

Refer to FIG. 2, FIG. 8 and FIG. 9. In some embodiments of the invention, the predetermined pair-data set is stored in the memory 1027. After the sensor 1022 penetrates into the user's skin, the first parameter value (current value) is obtained, and the first parameter value is input to the processor 1028. The processor 1028 calls the predetermined pair-data set from the memory 1027 and obtains the second parameter value by index based on the current value obtained by the sensor 1022. The in vivo analyte concentration is obtained and then sent by the transmitter 1023 to the remote equipment for user reference.

Considering the limited volume of the internal circuit 1024, the volume of the memory 1027 and the processor 1028 are limited, and it is impossible to store a large number of pair-data sets and perform a large number of data operations. In other embodiments of the invention, the memory 1027 and the processor 1028 are located in remote equipment, such as handheld machines, mobile phones, computers, etc. After the sensor 1022 obtains the current value, the transmitter 1023 sends it to the processor 1028 in the remote equipment. The processor 1028 calls the predetermined pair-data set from the memory 1027, obtains the second parameter value based on the current value index obtained by the sensor 1022, and obtains the in vivo analyte concentration for user reference.

In further embodiments of the invention, the memory 1027 is located in the remote equipment and the processor 1028 is located in the local internal circuit 1024. In further embodiments of the invention, the processor 1028 is located in the remote equipment and the memory 1027 is located in the local internal circuit 1024.

In some embodiments of the invention, the memory 1027 and the processor 1028 can be integrated in the same electronic device, such as CPU, MCU, etc. In some embodiments of the invention, the processor 1028 and the transmitter 1023 can be integrated in the same electronic device, such as a radio frequency chip. In some embodiments of the invention, the memory 1027 and the transmitter 1023 can be integrated in the same electronic device. In some embodiments of the invention, the transmitter 1023, the memory 1027, and the processor 1028 can be integrated in the same electronic device.

In other embodiments of the invention, the index function of the processor 1028 can also be realized by hardware. For example, a filter composed of a plurality of comparators with different high and low thresholds, each comparator is connected with a transmitter, and the transmission signal of the transmitter is the preset signal, which is associated with the blood glucose concentration information (second parameter value) or current value information (first parameter value). After the sensor 1022 obtains the current, the current enters the filter, and the current of different intensities can only pass through the comparator whose high threshold is higher than its value and whose low threshold is lower than its value. At the same time, the transmitter connected to the comparator is activated, and the transmitter sends the preset signal to the remote equipment.

Considering that the predetermined pair-data set is discrete, in some cases, the current value of sensor 1022 does not exist in the predetermined pair-data set, resulting in the failure indexing of processor 1028 and the inability to obtain the corresponding analyte concentration value. In view of this case, in the preferred embodiment of the invention, the processor 1028 needs to perform an interpolation operation when indexing. For example, when the current value detected by the sensor 1022 is x, the x value does not exist in the predetermined pair-data, but the x value exists between two adjacent first parameter values x₋₁ and x₊₁ recorded in the predetermined pair-data.

Therefore, the analyte concentration ƒ(x) corresponding to the current value x can be calculated by interpolation method:

$$\frac{x - {x}_{- 1}}{x - {x}_{+ 1}} = \frac{( f ( x ) - f ( {x}_{- 1} )}{( f ( x ) - f ( {x}_{+ 1} )}$$

In the embodiment of the invention, the higher the accuracy of the predetermined pair-data, the higher the accuracy of the result obtained by interpolation operation.

In view of the failure of the index of the processor 1028, in the preferred embodiment of the invention, the range of the analyte concentration ƒ(xₙ) is preset to be 30 mg/dL˜150 mg/dL during the test, and the accuracy is 0.1 mg/dL, for example, when ƒ(x₁)=30 mg/dL, ƒ(x₂)=30.1 mg/dL . . . and respectively obtain the current values x₁, x₂ . . . of the sensor 1022 at the corresponding concentration. When setting the predetermined pair-data set, the first parameter value is set as the range value of the average value of the adjacent first parameter values, for example, the current value corresponding to the analyte concentration ƒ(x₂) is

$$[ \frac{{x}_{1} + {x}_{2}}{2} , \frac{{x}_{2} + {x}_{3}}{2} ) ,$$

the current value corresponding to the analyte concentration ƒ(x₃) is

$$[ \frac{{x}_{2} + {x}_{3}}{2} , \frac{{x}_{3} + {x}_{4}}{2} ) …$$

and so on. It should be noted that the current values corresponding to the first analyte concentration ƒ(x₁) and the last analyte concentration ƒ(xₙ) of the pair-data set are

$$[ {x}_{1} , \frac{{x}_{1} + {x}_{2}}{2} ) and [ \frac{{x}_{n - 1} + {x}_{n}}{2} , {x}_{n} ] ,$$

the second pair-data set as shown in FIG. 10 is obtained. In this pair-data set, the first parameter value is continuous, and there will be no index failure. For example, when the current value detected by the sensor 1022 is x, and the processor 1028 judges that the value meets

$$\frac{{x}_{298} + {x}_{299}}{2} < x < \frac{{x}_{299} + {x}_{300}}{2} ,$$

the analyte concentration at this time can be obtained as ƒ(x₂₉₉) by index.

FIG. 11 is the flow chart of the use of the second pair-data set of the analyte detection system.

In the embodiment of the invention, the memory 1027 stores the predetermined pair-data set. After the sensor 1022 penetrates into the user's skin, it obtains the first parameter value (current value), inputs the first parameter value to the processor 1028, and the processor 1028 calls the predetermined pair-data set from the memory 1027 and determines the range in which the first parameter value falls, the second parameter value is obtained according to the falling range by index.

Based on the market demand, considering that the sensors 1022 need to be mass produced and used, there may be thousands or even tens of thousands of sensors in the same batch, and the manufacturing parameters and physical characteristics of each sensor will not be exactly the same. If each sensor is tested with high precision, such as 1201 tests for each sensor, it will consume a lot of production time of manufacturers. Considering this situation, an efficient sensor calibration scheme is also needed.

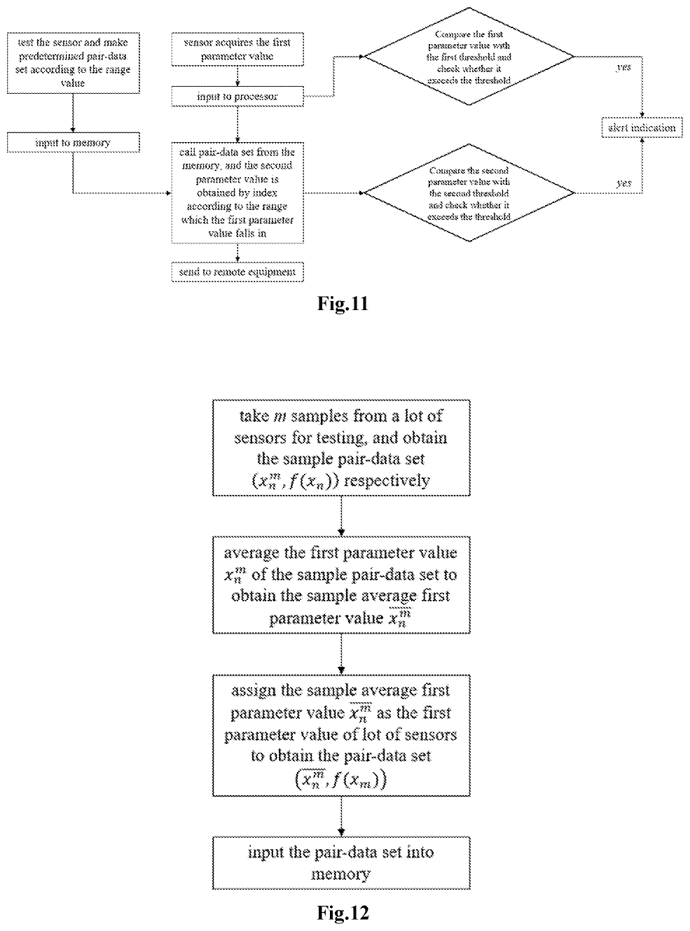

FIG. 12 is the flow chart of the first calibration scheme according to an embodiment of the invention.

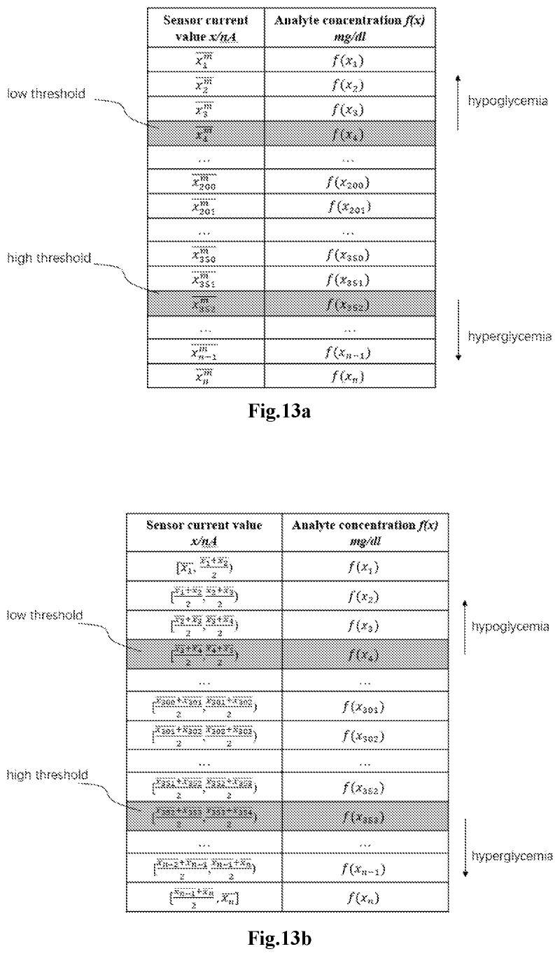

In the embodiment of the invention, m samples are taken from a lot of sensors, such as 10000 sensors, for testing, and the pair-data sets (xₙ^{m}, ƒ(xₙ) of each sample are obtained respectively, and average the first test parameter values xₙ¹˜xₙ^{m} of all sample data pairs to obtain the average first test parameter values x̅ₙ̅^̅{̅m̅}̅ of samples. Then, the average first test parameter value x̅ₙ̅^̅{̅m̅}̅ of the sample is given as the first parameter value of all sensors in this lot to obtain the average pair-data set (x̅ₙ̅^̅{̅m̅}̅, ƒ(xₙ) as shown in FIG. 13a, the average pair-data set is used as the predetermined pair-data set of the sensor and input into the memory 1027.

In the embodiment of the invention, the number m can be selected as 1/1000, 1/100, 1/50, 1/20 or 1/10 of the number of sensors in the same lot. The more samples taken, the higher the average pair-data accuracy, but the more test time it takes. In the preferred embodiment of the invention, the number m is selected as 1/50 of the number of sensors in the same lot.

Considering that the average first parameter value given to the lot is the discrete value, the processor 1028 will also fall in indexing.

In some embodiments of the invention, the processor 1028 needs to perform an interpolation operation when indexing. For example, when the current value detected by the sensor 1022 is x, the value x does not exist in the predetermined pair-data set, but the value is located in two adjacent average first parameter values x̅₋̅₁̅ and x̅₊̅₁̅ recorded in the predetermined pair-data set. Therefore, the analyte concentration ƒ(x) corresponding to the current value x can be calculated by interpolation method:

$$\frac{x - \overline{{x}_{- 1}}}{x - \overline{{x}_{+ 1}}} = \frac{( f ( x ) - f ( {x}_{- 1} )}{( f ( x ) - f ( {x}_{+ 1} )}$$

In other embodiments of the invention, when setting the predetermined pair-data set, the first parameter value of the sensor is set as the range value composed of the average value of the adjacent average first test parameter values, for example, the current value corresponding to the analyte concentration ƒ(x₂) is

$$[ \frac{\overline{{x}_{1}} + \overline{{x}_{2}}}{2} , \frac{\overline{{x}_{2}} + \overline{{x}_{3}}}{2} ) ,$$

the current value corresponding to ƒ(x₃) is

$$[ \frac{\overline{{x}_{2}} + \overline{{x}_{3}}}{2} , \frac{\overline{{x}_{3}} + \overline{{x}_{4}}}{2} ) …$$

and so on. It should be noted that the current values corresponding to the first analyte concentration value ƒ(x₁) and the last analyte concentration value ƒ(xₙ) of the pair-data set are

$$[ \overline{{x}_{1}} , \frac{\overline{{x}_{1}} + \overline{{x}_{2}}}{2} ) and [ \frac{\overline{{x}_{n - 1}} + \overline{{x}_{n}}}{2} , \overline{{x}_{n}} ]$$

respectively. The average range value pair-data set as shown in FIG. 13b is obtained. In this pair-data set, the average first test parameter value is continuous, and there will be no index failure. For example, when the current value detected by the sensor 1022 is x, the processor 1028 determines that the value x meets

$$\frac{\overline{{x}_{298}} + \overline{{x}_{299}}}{2} < x < \frac{\overline{{x}_{299}} + \overline{{x}_{300}}}{2} ,$$

the analyte concentration at this time can be obtained by index as ƒ(x₂₉₉).

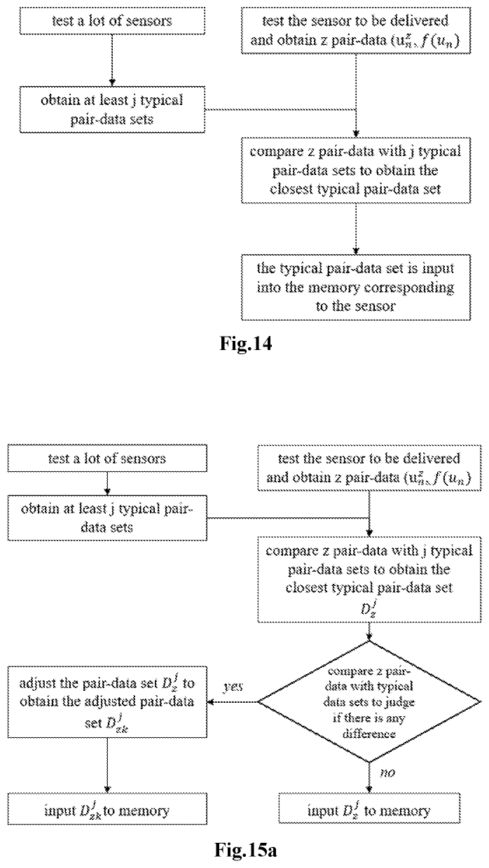

FIG. 14 is the flow chart of the second calibration scheme according to an embodiment of the invention.

Considering that in the sample calibration scheme, the manufacturer still needs to test a large number of samples with high precision, which will still consume more production time. In the embodiment of the invention, a lot of sensors (e.g. i) can be tested before production to obtain the predetermined pair-data set (xₙⁱ, ƒ(xₙ)) composed of the first test parameter value and the second parameter value. The number i of the lot can be 100, 1000, 10000 or more. The more the number, the more conducive it is to the classification and division of subsequent pair-data sets.

Because the second parameter values are consistent when testing the sensor, the predetermined pair-data set is summarized based on the first test parameter value to obtain the typical pair-data set Dⁱ:

$${D}^{i} = \{ ( ( {x}_{1}^{1} \sim {x}_{1}^{i} ) , f ( {x}_{1} ) ) ; ( ( {x}_{2}^{1} ∼ {x}_{2}^{i} ) , f ( {x}_{2} ) ) ; ( ( {x}_{3}^{1} ∼ {x}_{3}^{i} ) , f ( {x}_{3} ) ) … ( ( {x}_{n}^{1} \sim {x}_{n}^{i} ) , f ( {x}_{n} ) ) \}$$

The above typical pair-data set Dⁱ is classified according to the hold out method or cross validation method to obtain j typical pair-data sets D^{j}:

$${D}^{j} = \{ ( ( {x}_{1}^{1} \sim {x}_{1}^{j} ) , f ( {x}_{1} ) ) ; ( ( {x}_{2}^{1} ∼ {x}_{2}^{j} ) , f ( {x}_{2} ) ) ; ( ( {x}_{3}^{1} \sim {x}_{3}^{j} ) , f ( {x}_{3} ) ) … ( ( {x}_{n}^{1} ∼ {x}_{n}^{j} ) , f ( {x}_{n} ) ) \}$$

In the embodiment of the invention, the j typical pair-data sets can be 10, 20, 50, 100 or more. The more the number of typical pair-data sets, the more accurate the classification of pair-data sets, but also the greater the amount of subsequent calculation. The above j typical pair-data sets D^{j} can characterize the pair-data sets of all sensors.

In the embodiment of the invention, after obtaining the typical pair-data sets D^{j}, the above typical pair-data sets D^{j} is stored in the computer of the production line. During formal production, it is only necessary to test z pair-data (uₙ^{z}, ƒ(uₙ)), and select the closest typical pair-data set D_{z}^{j} to the z pair-data, the pair-data set D_{z}^{j} is stored in the memory 1027 corresponding to the sensor as the predetermined pair-data set of the sensor to be delivered. Compared with the sample calibration scheme, the typical pair-data set calibration scheme can further save production time and improve production efficiency.

In the embodiment of the invention, the typical pair-data set closest to z pair-data can be obtained by minimizing the sum of squares of differences. That is, input the first test parameter value uₙ^{z} of z pair-data into the computer, and the computer takes z first test parameter values uₙ^{z} into j typical pair-data sets D^{j} for calculation, and the typical pair-data set D_{z}^{j} of min(Σ(uₙ^{z}−xₙ^{j})²) is the closest typical pair-data set, which can most approximately represent the actual pair-data set of the sensor to be delivered. At the same time, the computer inputs the typical pair-data set into the memory corresponding to the sensor to be delivered as the predetermined pair-data set of the sensor to be delivered.

In the embodiment of the invention, the z pair-data to be tested can be 10, 50 or 100. The more pair-data tested, the closer the typical pair-data set found by calculation is to the actual pair-data set of the sensor to be delivered, but it also leads to more production time and larger calculation amount.

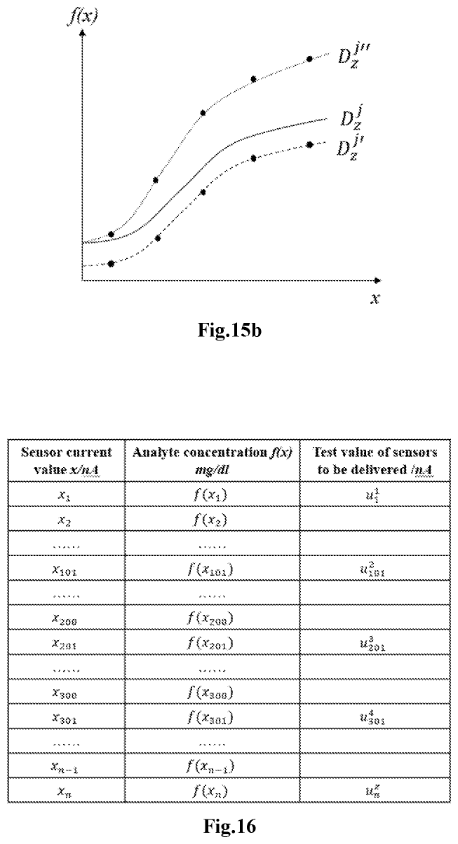

FIG. 15a is the flow chart of calibration based on the difference of physical characteristics of the sensor according to the embodiment of the invention; FIG. 15b is the schematic diagram of the visualization curve of the pair-data set in the coordinate system according to an embodiment of the invention.

Considering the differences in physical characteristics between sensors, such as film thickness, area of active enzyme layer, volume of active enzyme layer or electrode resistance, there is a fixed difference or linear difference between the actual pair-data set of the sensor to be delivered and the closest typical pair-data set or other predetermined pair-data set, as shown in FIG. 15b. In the FIG. 15b, the curve D_{z}^{j}′ and D_{z}^{j}″ represents the actual pair-data set of the sensor to be delivered, the curve D_{z}^{j} represents the closest typical pair-data set. D_{z}^{j}′ relative to D_{z}^{j} has a fixed difference, D_{z}^{j}″ relative to D_{z}^{j} has a linear difference.

Continue to refer to FIG. 15a. In the embodiment of the invention, after the closest typical pair-data set D_{z}^{j} is found in the sensor to be delivered, compare the tested z pair-data with the typical pair-data set to see if there is fixed difference or linear difference. If there is no difference, input the typical pair-data set D_{z}^{j} into memory; If there is difference, it is necessary to adjust the first parameter value in the typical pair-data set D to reduce the difference between the typical pair-data set and the actual pair-data set, and input the adjusted pair-data set D_{z}^{j} into the memory.

In the embodiment of the invention, the adjustment of the first parameter value in the typical pair-data set can be realized by the adjustment function xₜ:

$${x}_{t} = a * x + b$$

there,
a is the linear adjustment coefficient;
b is the fixed adjustment coefficient;
x is the first parameter value in the typical pair-data set before adjustment.

In the embodiment of the invention, the fixed adjustment coefficient b is calculated from the fixed difference between the actual pair-data set of the sensor and the typical pair-data set. The linear adjustment coefficient a is calculated from the linear difference between the actual pair-data set of the sensor and the typical pair-data set.

FIG. 16 is the schematic diagram of the pair-data set in the second calibration scheme of the embodiment of the invention.

In the embodiment of the invention, the z pair-data to be tested by the factory sensor are selected by equidistant distribution, as shown in FIG. 16.

In other embodiments of the invention, the z pair-data to be tested by the factory sensor are selected by random distribution.

Considering that during the use of the analyte detection system, with the increase of service time, due to the change of sensor enzyme layer activity, electrode oxidation and other factors, the predetermined pair-data set deviates from the actual pair-data set of the sensor, and this deviation will continue to change, so storing a fixed predetermined pair-data set in the memory may not meet the long-term use requirements of the sensor.

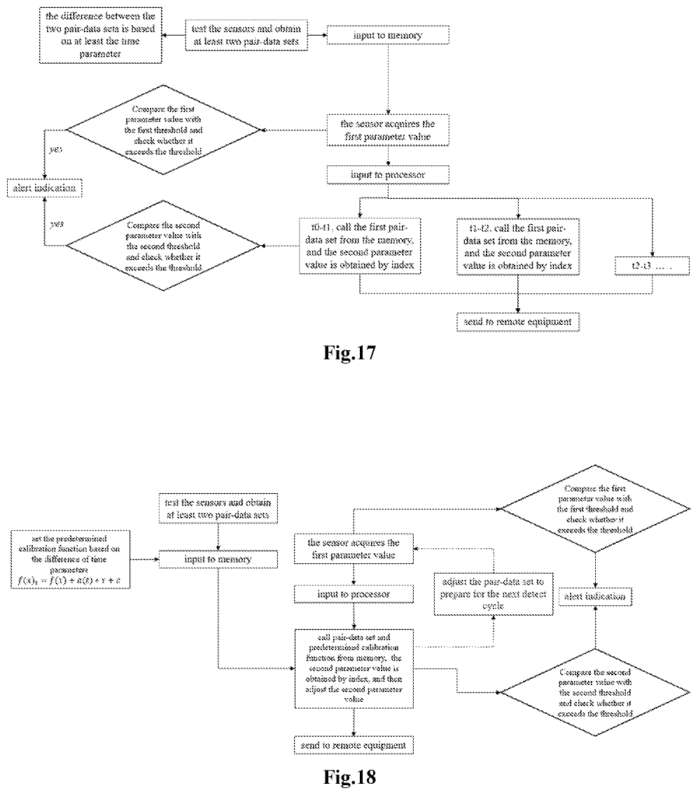

FIG. 17 is the flow chart of the first method of time parameter difference calibration according to an embodiment of the invention.

In the embodiment of the invention, the sensor to be delivered is tested at t0 time to obtain the first predetermined pair-data set Dₜ₀ at t0 time, and then tested again at t1 time to obtain the second predetermined pair-data set Dₜ₁ at t1 time . . . so as to repeatedly obtain a plurality of predetermined pair-data sets based on the difference of time parameters and input them into the memory corresponding to the sensor to be delivered. Meanwhile, the processor is programmed to call the first predetermined pair-data set Dₜ₀ from the memory at t0-t1 time. After the sensor obtains the first parameter value, the processor obtains the second parameter value based on the first parameter value by index in the first predetermined pair-data set Dₜ₀. And then the second predetermined pair-data Dₜ₁ is called from the memory at t1-t2 time, and the sensor obtains the first parameter value, the processor obtains the second parameter value based on the first parameter value by index in the second predetermined pair-data Dₜ₁, . . . . Until the service life of the sensor is terminated or the analyte detection device stops working.

In the embodiment of the invention, the number of the predetermined pair-data sets input to the memory is determined by the test interval Δt and the service life T of the sensor. For example, when the service life T of the sensor is 14 days and the test interval Δt is 1 day, the number of the predetermined pair-data sets is T/Δt=14.

In some embodiments of the invention, a lot of predetermined pair-data sets based on time parameter differences may be pair-data sets as shown in FIG. 7 or FIG. 13a, which are indexed by interpolation when used.

In some embodiments of the invention, a lot of predetermined pair-data sets based on time parameter differences can also be pair-data sets as shown in FIG. 10 or FIG. 13b, which are indexed based on the range in which the first parameter value falls.

In some embodiments of the invention, a lot of predetermined pair-data sets based on time parameter differences can also be typical pair-data as shown in FIG. 16. J typical pair-data sets are set respectively in t0-t1, t1-t2, t2-t3 and other time periods, and then the closest typical pair-data set of the sensor to be delivered is found in each time period through calculation, and then stored in the memory 1027 corresponding to the sensor. Accordingly, the processor 1028 is programmed to call typical pair-data sets and index in each time period.

FIG. 18 is the second flow chart of time parameter difference calibration according to the embodiment of the invention.

In the embodiment of the invention, the memory 1027 also stores the predetermined calibration function ƒ(x)ₜ which is based on the difference of time parameters:

$${f ( x )}_{t} = f ( x ) + a ( t ) * x + c$$

there,
ƒ(x)ₜ is the second parameter value after adjustment;
ƒ(x) is the second parameter value before adjustment;
a(t) is the predetermined calibration scale factor, which is related to the service time of the sensor;
c is the predetermined calibration constant.

In some embodiments of the invention, the predetermined calibration scale coefficient and the predetermined calibration constant are obtained from the test of the sensor to be delivered. In other embodiments of the invention, the predetermined calibration proportion coefficient and the predetermined calibration constant are obtained from the samples of a lot of sensors, and then the sample data are averaged and given to the lot.

Referring to FIG. 18, in the embodiment of the invention, when using the analyte detection system, the sensor 1022 obtains the first parameter value and inputs it to the processor 1028. The processor 1028 calls the pair-data set and the predetermined calibration function based on the time parameter difference from the memory 1027, and the second parameter value is obtained in the pair-data set by index based on the first parameter value, and the obtained second parameter value is adjusted through the predetermined calibration function to obtain the adjusted second parameter value and send it to the remote device.

In other embodiments of the invention, the sensor 1022 obtains the first parameter value and inputs it to the processor 1028. The processor 1028 calls the pair-data set and the predetermined calibration function based on the time parameter difference from the memory 1027, the second parameter value is obtained in the pair-data set by index based on the first parameter value and output to the remote device, and then adjusts the pair-data set according to the predetermined calibration function, the adjusted pair-data set is stored in the memory 1027 as the pair-data set to be called in the next detection cycle. In some embodiments of the invention, the adjustment of the pair-data set is to adjust the first parameter value and keep the second parameter value unchanged. In other embodiments of the invention, the adjustment of the pair-data set is to adjust the first parameter value and the second parameter value at the same time. In other embodiments of the invention, the adjustment of the pair-data set is to adjust the second parameter value and keep the first parameter value unchanged.

With reference to FIGS. 7, 9, 10, 11, 13a, 13b, 17 and 18. In an embodiment of the invention, the first parameter threshold is set in the processor 1028 and corresponds to the first parameter value, such as a current threshold or a voltage threshold. The first parameter threshold comprises a high threshold with a higher value and a low threshold with a lower value, and the region between the high threshold and the low threshold is the normal interval. When the sensor 1022 obtains the first parameter value and inputs it to the processor 1028, the processor 1028 compares the first parameter value with the first parameter threshold. If the first parameter value exceeds the high threshold, it is judged that there is a risk of hyperglycemia. If the first parameter value is lower than the low threshold, it is judged that there is a risk of hypoglycemia. When the processor 1028 determines that there is a risk of hyperglycemia or hypoglycemia, it outputs an alert indication.

In other embodiments of the invention, the second parameter threshold is set in the processor 1028 and corresponds to the second parameter value, such as blood glucose concentration threshold. The second parameter threshold comprises a higher high threshold and a lower low threshold, and the region between the high threshold and the low threshold is the normal interval. After the processor 1028 obtains the second parameter value by index, the processor 1028 compares the second parameter value with the second parameter threshold. If the second parameter value exceeds the higher high threshold, it is determined that there is a risk of hyperglycemia. If the second parameter value is lower than the lower low threshold, it is determined that there is a risk of hypoglycemia. When the processor 1028 determines that there is a risk of hyperglycemia or hypoglycemia, it outputs an alert indication.

In the embodiment of the invention, the alert indication can be processed by the local internal circuit 1024 or by the remote equipment. After the alert indication is processed, it will prompt the user or other monitoring personnel in one or more forms such as lighting, sound and vibration.

In the embodiment of the invention, the first parameter threshold or the second parameter threshold in the processor 1028 can be set by the user or non-user. For example, the first parameter threshold or the second parameter threshold is set in the processor 1028 at the factory, or the first parameter threshold or the second parameter threshold is set by other guardians.

To sum up, the invention provides a calibration free analyte detection device. At least one predetermined pair-data set composed of the first parameter value and the second parameter value and the predetermined calibration function based on the time parameter are pre stored in the memory. After the sensor penetrates into the user's skin to obtain the first parameter value, the processor calls the predetermined pair-data set and the predetermined calibration function from the memory and obtains the second parameter value based on the first parameter value by index, the predetermined pair-data set is adjusted through the predetermined calibration function for the next detection and retrieval. The predetermined pair-data set is adjusted with the use time, which reduces the error of the second parameter value and improves the reliability of the analyte detection device.

Although some specific embodiments of the invention have been detailed through examples, technicians in the field should understand that the above examples are for illustrative purposes only and are not intended to limit the scope of the invention. Persons skilled in the field should understand that the above embodiments may be modified without departing from the scope and spirit of the invention. The scope of the invention is limited by the attached claims.

1. An analyte detection device, comprising:
a shell;
a sensor comprising an internal part and an external part, wherein the internal part is used to penetrate into a subcutaneous skin to obtain a first parameter value;
a memory in which at least a predetermined pair-data set composed of the first parameter value and a second parameter value and a predetermined calibration function based on time parameters are pre stored, wherein the second parameter value is associated with an in vivo analyte parameter information;
a processor programmed to call the predetermined pair-data set and the predetermined calibration function from the memory, obtaining the second parameter value based on the first parameter value by an index, and the predetermined pair-data set is adjusted by predetermined calibration function, wherein the predetermined pair-data set is callable by the processor in a next detection;
a transmitter used to transmit the first parameter value and/or the second parameter value to a remote equipment; and
a battery, which is used to provide electric energy.

2. According to the analyte detection device mentioned in claim 1, wherein the first parameter value is a current value or a voltage value.

3. According to the analyte detection device mentioned in claim 1, wherein the second parameter value comprises a blood glucose concentration value.

4. According to the analyte detection device mentioned in claim 1, wherein the predetermined pair-data set is at least partially derived from in vivo tests.

5. According to the analyte detection device mentioned in claim 1, wherein the predetermined pair-data set is at least partially derived from in vitro tests.

6. According to the analyte detection device mentioned in claim 1, wherein at least some of pair-data in the predetermined pair-data set is adjustable based on a physical characteristics of the sensor.

7. According to the analyte detection device mentioned in claim 6, wherein the physical characteristics of the sensor comprise at least one of a membrane thickness, an active enzyme area, an active enzyme volume or a resistance of an electrode.

8. According to the analyte detection device mentioned in claim 6, wherein an adjustment of the at least some of the of pair-data is a fixed value.

9. According to the analyte detection device mentioned in claim 6, wherein an adjustment of the at least some of the of pair-data is changed in a linear manner.

10. According to the analyte detection device mentioned in claim 1, wherein the first parameter value or the second parameter value is set with at least one threshold, wherein when the first parameter value or the second parameter value exceeds the threshold, the remote equipment sends an alert indication.

11. According to the analyte detection device mentioned in claim 12, wherein the threshold is set by a user or a non-user.

12. According to the analyte detection device mentioned in claim 1, wherein the second parameter value is obtained by interpolating the first parameter value.

13. According to the analyte detection device mentioned in claim 1, wherein the first parameter value in the first predetermined pair-data set or the second predetermined pair-data set is a continuous range interval, wherein the second parameter value is obtained by judging the range interval in which the first parameter value falls with indexing.

14. According to the analyte detection device mentioned in claim 1, wherein the transmitter, the memory, the sensor, the processor and the battery are located in the shell.

15. According to the analyte detection device mentioned in claim 1, wherein the transmitter, the sensor and the battery are located in the shell, and the memory and/or the processor are located in the remote equipment.

16. According to the analyte detection device mentioned in claim 1, wherein at least two of the transmitter, the processor and the memory are integrated.

17. A calibration method for analyte detection, comprising:
providing a memory, wherein at least a predetermined pair-data set composed of a first parameter value and a second parameter value and a predetermined calibration function based on time parameters are pre-stored in the memory, wherein the second parameter value is associated with an in vivo analyte parameter information;
providing a sensor comprising an internal part and an external part, wherein the internal part is penetrated into a subcutaneous to obtain the first parameter value;
providing a processor programmed to obtain the first parameter value from the sensor, calling the predetermined pair-data set and the predetermined calibration function from the memory, and obtaining the second parameter value based on the first parameter value by an index, wherein the predetermined pair-data set is adjusted by the predetermined calibration function, wherein the predetermined pair-data set is callable by the processor in a next detection; and
providing a transmitter sending the first parameter value and/or the second parameter value to a remote equipment.
